(19) United States
(12) Patent Application Publication
(10) Pub. No.: US 2024/0316078 A1
(43) Pub. Date: Sep. 26, 2024

(54) Treatment of Neuropathic Sensitization Disorders

(71) Applicant: IVIEW Therapeutics, Inc., Cranbury, NJ (US)

(72) Inventor: Edward Tak Wei, Berkeley, CA (US)

(21) Appl. No.: 18/260,908

(22) PCT Filed: Jan. 10, 2022
(86) PCT No.: PCT/US2022/011874

§ 371 Date: Jul. 10, 2023

Related U.S. Application Data
(60) Provisional application No. 63/205,848, filed on Jan. 11, 2021.

(51) Int. Cl. A61K 31/66; A61K 9/00; A61P 25/00; A61P 27/02

(52) U.S. Cl. CPC A61K 31/66; A61K 9/0048; A61P 25/00; A61P 27/02

(57) ABSTRACT

Among others, the present invention provides a method for treating a neuropathic ocular pain disorder in a subject in need thereof, comprising: topically applying a therapeutically effective amount of a 1-di-isopropyl-phosphinoyl-alkane (DIPA) compound onto an ocular surface of the subject for at least one week, wherein the DIPA compound is dissolved in a liquid vehicle which is adapted for focused delivery of the DIPA compound to the ocular surface.

### CROSS-REFERENCE TO RELATED APPLICATION

This application claims priority to U.S. Provisional Application No. 63/205,848, filed Jan. 11, 2021, the contents of which are incorporated herein by reference in their entirety.

### BACKGROUND OF THE INVENTION

Sir Charles Sherrington defined pain in about 1900 as “the psychical adjunct of an imperative protective reflex.” The modern definition of “pain” is by the International Association for the Study of Pain (IASP) as an unpleasant sensory and emotional experience associated with, or resembling that associated with, actual or potential tissue damage.

Updated notes accompanying the new definition are: pain is always a personal experience that is influenced to varying degrees by biological, psychological, and social factors; pain and nociception are different phenomena; and pain cannot be inferred solely from activity in sensory neurons.

Through their life experiences, individuals learn the concept of pain. A person's report of an experience as pain should be respected.

Although pain usually serves an adaptive role, it may have adverse effects on function and social and psychological well-being.

Verbal description is only one of several behaviors to express pain; inability to communicate does not negate the possibility that a human or a nonhuman animal experiences pain.

In the Sherrington and the IASP definitions, there is recognition and emphasis on the psychical and experiential aspects of pain, namely, that it is an event that is perceived by the mind.

Advances in neurophysiology and molecular biology have accelerated an understanding of the mechanisms of pain. It is now recognized that pain is activated by an increased discharge of unmyelinated small-diameter sensory fibers called polymodal C fibers. Pain is categorized as nociceptive or neuropathic. Nociceptive pain is caused by cell injury, such as trauma, inflammation, and immune disorders. Neuropathic pain is caused by damage to the nerve fibers that transmit the pain signals. Sensations that may accompany pain are irritation, pruritus (itch), and a sense of malaise and disaffection. In this application, the psychical adjuncts of nociception are also categorized as “sensory discomfort” or dysesthesia.

Chronic pain is defined as persistent or recurrent pain lasting longer than three months. There are optional specifiers such as pain severity for each patient, which can be graded on intensity, pain-related distress, and impairment of function. The types of chronic pain include cancer pain, postsurgical and posttraumatic pain, musculoskeletal pain, headache and orofacial pain, visceral pain, and neuropathic pain.

Neuropathic pain may be spontaneous or evoked, as an increased response to a painful stimulus (hyperalgesia) or as a painful response to a normally nonpainful stimulus (allodynia). The increased amplification of pain, in hyperalgesia or hyper-responsiveness in allodynia, is termed “sensitization”, and this can occur in the peripheral nerves (peripheral sensitization) or in the central nervous system (central sensitization). The IASP now standardizes these terms. The term “hypersensitivity” is not used for pain descriptions because traditionally hypersensitivity refers to undesirable reactions produced by the immune system, including allergies and autoimmunity.

As used herein, “sensitization” refers to increased responsiveness of nociceptive neurons to their normal input, and/or recruitment of a response to normally subthreshold inputs.

“Central sensitization” refers to increased responsiveness of nociceptive neurons in the central nervous system to their normal or subthreshold afferent input.

“Peripheral sensitization” refers to increased responsiveness and reduced threshold of nociceptive neurons in the periphery to the stimulation of their receptive fields.

Neuropathic ocular pain (NOP) refers to pain from the ocular surface (defined as the epithelia of the cornea, limbus, conjunctiva, and eyelid margins). One mechanism of NOP comes from repeated direct damage to corneal nerves. Aberrant regeneration of nerve endings with upregulation of nociceptors may be responsible for peripheral sensitization. The persistent pain may then cause central sensitization and distress. NOP can occur after eye injury has healed and in the absence of detectable anatomic disruption—the so-called corneal “pain without stain.” NOP has been called corneal neuropathy, corneal neuralgia, kertaoneuralgia, and corneal allodynia. Today chronic pain has a more standardized terminology in the 11th Edition of the International Classification of Diseases, where the classification of chronic pain is located in section MG30 of Chapter 21. NOP is classified as chronic neuropathic pain.

NOP has a severe negative impact on the quality of life of patients. The sensations of pain, sensitivity to light, and irritation are intense, constant, and persistent, and impair ability to perform daily activities such as watching TV, reading, driving, and working. Physical and social functions diminish, and there is distress. A large group of NOP patients suffer from chronic dry eye syndromes that are not responsive to conventional treatment. But NOP can occur without the signs of dry eye disease, such as changes in the rates of tear secretion or changes in the quality or stability of the tears (e.g. from Meibomian gland dysfunction). Especially difficult to treat conditions are NOP from post-refractive or cataract surgery. Patients become desperate and suicidal because pain is persistent, intractable, and dominates the psyche. A well-known recent case is J. S., a 35-year old TV meteorologist from Detroit and mother of two young children who committed suicide after NOP from Lasik surgery. Thorough and up-to-date discussions of NOP are found in papers by Anat Galor of the University of Miami, FL (Galor, A. et al., The Ocular Surface, 2018, 16, 31-44; Mehra D., Anat Galor, Ophthalmology and Therapy, 2020, 9 (3): 427-47).

By definition, chronic neuropathic pain is a condition that has lasted longer than three months. For NOP patients, this usually means that everything has been tried in a three month period, but with limited if any success. Ocular surface treatment, for example, with artificial tears, ointments, and gels, are recommended. These are followed by punctal plugs, topical and systemic antibiotics, anti-inflammatory steroids, and anti-inflammatory drugs such as cyclosporine and lifitegrast. Nerve growth factors and autologous serum are speculative procedures for neuro-regenerative therapy. Another course of action is to administer drugs that affect the central nervous system, such as antidepressants (e.g., amitriptyline, nortriptyline), anticonvulsants (e.g., carbamazepine), NSAIDS, tramadol, and gabapentin/pregabalin, all with variable success. If NOP is associated with migraine, treatment of the migraine may help alleviate the NOP. There is a need for new and effective treatment of NOP.

### SUMMARY OF THE INVENTION

The present invention provides a method and a topical medication for treating a neuropathic ocular pain disorder in a subject in need.

In one aspect, the present invention provides a method for treating a neuropathic ocular pain disorder in a subject in need thereof, comprising: topically applying a therapeutically effective amount of a 1-di-isopropyl-phosphinoyl-alkane (DIPA) compound onto an ocular surface of the subject. The synthesis and the receptor bioassays of the DIPA are described in U.S. Pat. No. 10,195,217 and incorporated herein by reference. The DIPA is applied to an ocular surface for at least one week, wherein the DIPA compound is dissolved in a liquid vehicle, and wherein the liquid vehicle is adapted for focused delivery of the DIPA compound to the ocular surface.

In some embodiments, the DIPA compound is dissolved in the liquid vehicle at a concentration therein of 0.5 to 5 mg/ml and the liquid vehicle delivers the DIPA compound to the ocular surface.

In some embodiments, the liquid vehicle is an aqueous solution.

In some embodiments, the liquid vehicle is water or isotonic saline.

In some embodiments, the DIPA compound is dissolved in the liquid vehicle at a concentration of 0.5 to 5 mg/ml.

In some embodiments, the DIPA compound dissolved in the liquid vehicle is delivered to the ocular surface of the subject with a wipe.

In some embodiments, the DIPA compound dissolved in the liquid vehicle is applied 4 times a day to the ocular surface of the subject.

In some embodiments, the DIPA compound is

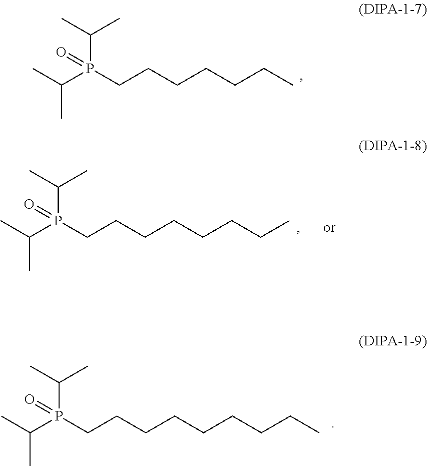

In some embodiments, the neuropathic ocular pain disorder is caused by dry eye disease.

In some embodiments, the neuropathic ocular pain disorder is caused by eye surgery.

In some embodiments, the neuropathic ocular pain disorder is caused by trauma to an eye.

In a second aspect, the present invention provides a topical medication for treating a neuropathic ocular pain disorder in a subject in need thereof, comprising an aqueous solution containing a therapeutically effective amount of a DIPA compound, which can be DIPA-1-7, DIPA-1-8, or DIPA-1-9 (i.e., 1-[diisopropyl-phosphinoyl]-nonane).

In some embodiments, concentration of the DIPA compound in the aqueous solution is 0.5 to 5 mg/mL.

In some embodiments, the neuropathic ocular pain disorder is caused by dry eye disease.

In some embodiments, the neuropathic ocular pain disorder is caused by eye surgery.

In some embodiments, the neuropathic ocular pain disorder is caused by trauma to an eye.

In a third aspect, the present invention provides use of a DIPA compound (e.g., DIPA-1-7, DIPA-1-8, or DIPA-1-9) for manufacturing a medicament for treating a neuropathic ocular pain disorder in a subject in need thereof, wherein the medicament comprises a therapeutically effective amount of the DIPA compound (e.g., DIPA-1-7, DIPA-1-8, or DIPA-1-9) and a liquid vehicle, wherein the liquid vehicle is adapted for focused delivery of the DIPA compound to an ocular surface of the subject.

These and other features, aspects, and advantages of the present invention will become better understood with reference to the following description and appended claims.

### BRIEF DESCRIPTIONS OF THE DRAWINGS

The embodiments of the application will now be described in greater detail with reference to the attached drawings in which:

FIG. 1 demonstrates a method of topical application of the gauze containing cryosim-3, which targets TRPM8 on the eyelid margin.

FIG. 2 is a schematic illustrating the mechanism of action of the TRPM8 agonist in relieving ocular pain in patients with dry eye.

### DETAILED DESCRIPTION OF THE INVENTION

In the Summary Section above and the Detailed Description Section, and the claims below, reference is made to particular features of the invention. It is to be understood that the disclosure of the invention in this specification includes all possible combinations of such particular features. For example, where a particular feature is disclosed in the context of a particular aspect or embodiment of the invention, or a particular claim, that feature can also be used, to the extent possible, in combination with and/or in the context of other particular aspects and embodiments of the invention, and in the invention generally.

In one aspect, the present invention provides a method for treating a neuropathic ocular pain disorder in a subject in need thereof, comprising: topically applying a therapeutically effective amount of a 1-di-isopropyl-phosphinoyl-alkane (DIPA) compound onto an ocular surface of the subject for at least one week, wherein the DIPA compound is dissolved in a liquid vehicle, and wherein the liquid vehicle is adapted for focused delivery of the DIPA compound to the ocular surface.

In a second aspect, the present invention provides a topical medication for treating a neuropathic ocular pain disorder in a subject in need thereof, comprising: an aqueous solution containing a therapeutically effective amount of a DIPA compound (e.g., DIPA-1-7, DIPA-1-8, or DIPA-1-9).

In a third aspect, the present invention provides use of a DIPA compound (e.g., DIPA-1-7, DIPA-1-8, or DIPA-1-9) for manufacturing a medicament for treating a neuropathic ocular pain disorder in a subject in need thereof, wherein the medicament comprises a therapeutically effective amount of the DIPA compound (e.g., DIPA-1-7, DIPA-1-8, or DIPA-1-9) and a liquid vehicle, wherein the liquid vehicle is adapted for focused delivery of the DIPA compound to an ocular surface of the subject.

Patients with a neuropathic ocular pain disorder experience neuropathic ocular pain (NOP). Neuropathic ocular pain (NOP) refers to pain from the ocular surface (defined as the epithelia of the cornea, limbus, conjunctiva, and eyelid margins). In some embodiments, the neuropathic ocular pain disorder is caused by dry eye disease. In some embodiments, the neuropathic ocular pain disorder is caused by eye surgery. In some embodiments, the neuropathic ocular pain disorder is caused by trauma to an eye.

Neurobiology of the Ocular Surface and Mechanism of Action: approximately 200 million years ago, certain living organisms acquired the ability to control metabolic heat production (endothermy) and to maintain a constant internal body temperature (homeothermy) (McNab, B. K. The evolution of endothermy in the phylogeny of mammals. American Naturalist 112: 1-21, 1978.). From a “cold-blooded” to a “warm-blooded” physiology, this evolutionary transition enabled such species to better adapt and survive in a variable environment. Associated with this change was developing and refining sensory systems to monitor and to control body temperature, especially on the eyes and in the upper respiratory tract, and to regulate drinking, thirst, and tear secretion. Coolness is a pervasive neuronal signal from the organism's surfaces such as the eyes, face, nose, ears, and neck. For example, from mammals' facial skin, about 92% of the thermoceptive input is from cold neurons. These neurons are tonically active at 15-18° C. (Hutchison, W. D. et al., J. Neurophysiol. 77: 3252-3266, 1997; Takashima, Y. et al., J. Neurosci., 27, 14147-14157, 2007).

The principal detector of coolness and cold is the integral membrane protein known as TRPM8 (Bautista, D. M. et al., Nature 448: 204-208, 2007). Another receptor that responds to lower temperatures is TRPA1. The anatomical architecture of the neurons containing TRPM8 has been mapped in mice (Dhaka et al., J. Neurosci. 28: 566-575, 2008; Schecterson et al., Molecular Vision 26:576-587, 2020). TRPM8-containing nerve fibers in the periphery are located in the epidermis' surface layers and project to superficial layers of the spinal cord and brainstem. Nerve fiber endings on the cornea and eyelids have also been mapped. The TRPM8 neuronal system is distinctly segregated from nociceptive neurons belonging to the C-fiber category. The TRPM8 nerve fibers are mostly myelinated and categorized as A-δ based on conduction velocity.

The TRPM8 peripheral cool/cold afferents were first carefully described in classical studies by Hensel. He mapped the density of “cold spots” on the body where the discrete application of cold could be associated with specific nerve fiber discharges. Thermosensation is tightly linked to perception and biological response systems. Thus, a hot shower is comfortable at 40° C., but at 43.4° C., the individual seeks to escape the heat. The 43.4° C. is also the point for the activation of heat/pain receptors and C-fiber discharge, for leakage of plasma contents from post-capillary venules, and the beginning of deranged cellular oxygen consumption. The same precise discrimination also occurs for cool/cold sensations. Thus, at about 18° C., individuals began to complain of cold, put on more clothes, and turn on the thermostat. Animals, in experimental situations, readily detect temperature differences of ±1° C. Chemical agents also select for ranges of cooling intensity. Some are mildly cool and tingling, others are refreshing cool, and yet others are just cold.

The antinociceptive properties of physical coolness/cold on the body's surfaces are to reduce irritation, itch, and pain. Thus, air-conditioning, cold water, and ice can be used to relieve sensory discomforts from heat, trauma, pain, and certain types of inflammation. The heat withdrawal transfer necessary for coolness/cold can be achieved with gas, liquid, or solid materials and utilizes mechanisms of evaporation, convection, or conduction of energy.

In the brain, there is modulated interaction among inputs from neurons, both nociceptive and non-nociceptive. There is also precise topographical recognition of the origin of the input. Witness the precise identification of the pain from a small pin or the havoc caused by an ingrown eyelash (trichiasis) or an ingrown toenail. The organism's normal function can be totally disrupted, so it is expected that neuropathy of the ocular surface nerves causes severe effects. The pharmacological strategy here is to use TRPM8 nerve input to gate and break the central sensitization of nociceptive perception in NOP. By interfering with the perception of noxious stimuli, an individual's psyche and anxiety about pain are diminished, with an overall improvement in the chronic pain state. The stated hypothesis is that cool/cold signals via AS-fiber are utilized to break the amplification of noxious signals and their subsequent pathogenesis.

Without being limited by theory, an analogy of the mechanism proposed here is as if there were three telephone lines in the tissues, each with a different dialing mechanism and cable conduction system. One is for touch and pressure that is fast conducting. One for coolness and cold that is somewhat slower (Aδ conducts at about 2 to 6 meters/sec). One for irritation, itch, and pain that conducts slowly (<2 meters/sec, primarily C-fibers). In the analogy, one of two telephone lines interferes with the other's signaling, but at the central exchange. It is proposed of using a compound of this discovery, 1-diisopropyl-phosphinoyl-nonane (Cryosim-3) or 1-diisopropylphosphinoyl-octane (Cryosim-2), as the dialing mechanism for stimulating the telephone line responsible for signals of coolness and cold. Using this telephone line, the generated signals are anticipated to diminish amplification of the noxious signals from the C-fiber line and to have a salutary effect on chronic pain.

Based on the above considerations and the data in the Example 1, it is proposed that TRPM8 agonists initiate Aδ-fiber input into the central nervous system and alters the flow of nociceptive information. This mode of action is indirect because there is no interference with the generation or transmission of the input of signals from the nociceptors. It should be noted that cool/cold signals are tonically active at 18° C. and constitute 92% of the thermoceptive input from the face's skin. In dorsal horn neurons, 50% of the TRPM8 cells are active in the narrow temperature range of 18 to 19° C. Thus, a slight increase in discharge frequency of TRPM8, because of its sheer volume, will dominate sensory transmission and integration in the central nervous system. This flood of cooling signals can alter increased sensitization of the nociceptive system.

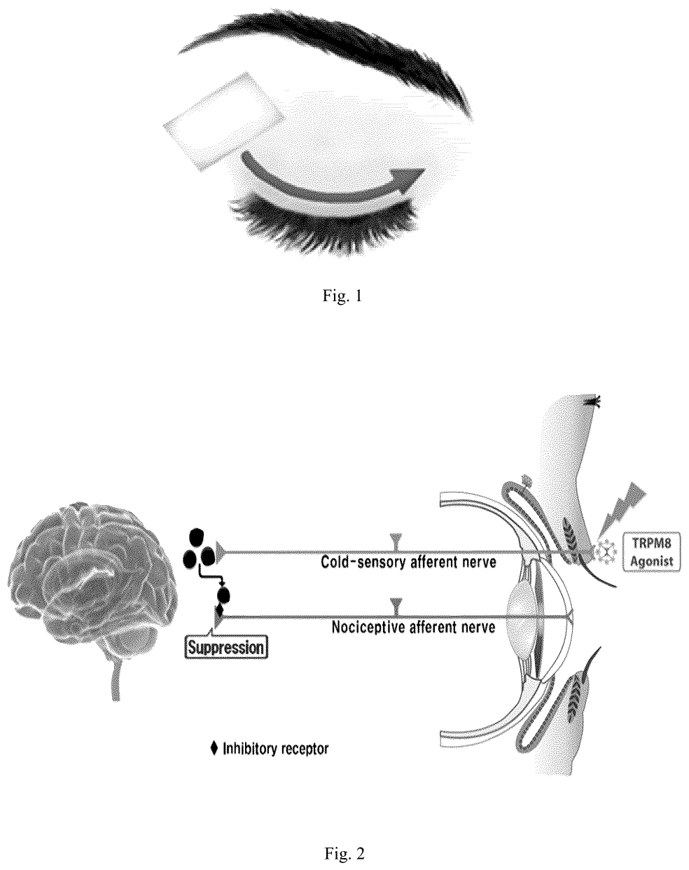

For cryosims, the drug delivery method is topical and focused on the receptive field containing the nociceptors or on the immediately adjacent sensory fields. In some embodiments, the DIPA compound dissolved in the liquid vehicle is delivered to the ocular surface of the subject with a wipe. In some embodiments, the DIPA compound dissolved in the liquid vehicle is applied 1, 2, 3, or 4 times a day to the ocular surface of the subject. The mode of antineuropathic action is indirect, that is, there is no direct effect on transmission of the signals. Cryosim-3 is designed to work on non-keratinized tissues. Its receptive field is the nerve endings of the trigeminal nerve's ophthalmic branches, especially in the receptive fields of the supraorbital nerve. A schematic of this method is shown in FIG. 1 and FIG. 2. The cooling agent is applied to the receptive field of TRPM8 neurons on the ocular margins (FIG. 1). The dedicated TRPM8 fibers are in the afferents of the supraorbital nerve (green). When these signals reach the trigeminal nuclei in the brainstem the cooling signals intercept and inhibit nociceptive signals transmitted via the afferents of the ciliary nerve (red) [FIG. 2].

The methods for selecting and synthesizing the cryosims used here are described in Wei Ser. No. 16/350,559, US 2019/0105335, published Apr. 11, 2019. The preferred embodiment for the practice of this invention is 1-[Diisopropyl-phosphinoyl]-nonane (synonyms: Cryosim-3, 1-diisopropyl-phosphorylnonane, CAS Registry No. 1503744-37-8-7). Cryosim-3 is a synthetic molecule available at >97% purity from Phoenix Pharmaceuticals, Burlingame, Calif. USA.

In some embodiments, the DIPA compound is a pharmaceutically acceptable salt, polymer, ester, or acid thereof.

In some embodiments, the DIPA compound may be mixed with other ingredients, such as other active agents, preservatives, buffering agents, diluent, salts, a pharmaceutically acceptable carrier, or other pharmaceutically acceptable ingredients.

As used herein, a “diluent” refers to an ingredient in a pharmaceutical composition that lacks pharmacological activity but may be pharmaceutically necessary or desirable. For example, a diluent may be used to increase the bulk of a potent drug whose mass is too small for manufacture and/or administration. It may also be a liquid for the dissolution of a drug to be administered by injection, ingestion or inhalation. A common form of diluent in the art is a buffered aqueous solution such as, without limitation, phosphate buffered saline that mimics the composition of human blood.

As used herein, a “carrier” refers to a compound that facilitates the incorporation of a compound into cells or tissues. For example, without limitation, dimethyl sulfoxide (DMSO), Ethanol (EtOH), or PEG400 is a commonly utilized carrier that facilitates the uptake of many organic compounds into cells or tissues of a subject.

As used herein, the terms “individual,” “patient,” or “subject” are used interchangeably. None of the terms require or are limited to situation characterized by the supervision (e.g. constant or intermittent) of a health care worker (e.g. a doctor, a registered nurse, a nurse practitioner, a physician's assistant, an orderly, or a hospice worker.

As used herein, a “therapeutically effective amount” refers to a sufficient amount of a DIPA compound, at a reasonable benefit/risk ratio applicable to treating a neuropathic ocular pain disorder in a subject in need thereof. It will be understood, however, that the total daily usage of the DIPA compound may be decided by the attending physician or personal coach within the scope of sound medical judgment. The specific effective dose level for any particular subject will depend upon a variety of factors including the other disorder being treated and the severity of the disorder; the specific composition employed, the age, body weight, general health, sex and diet of the subject; the time of administration, route of administration, and rate of excretion of the DIPA compound employed; the duration of the administration; drugs used in combination or coincidental with the DIPA compound; and like factors well known in the medical arts or sports science. In addition, a “therapeutically effective amount” is the amount that will elicit the biological or medical response of a tissue, system, or subject that is being sought by a researcher or clinician.

One of skill in the art recognizes that an amount may be considered “effective” even if the condition is not totally eradicated or prevented, but it or its symptoms and/or effects are improved or alleviated partially in the subject. Various indicators for determining the effectiveness of a method are known to those skilled in the art for treating a neuropathic ocular pain disorder in a subject in need thereof.

As used herein, “focused delivery” means a “site-specific delivery” of a low volume of liquid to a designated anatomic site. It is expected that the active ingredient would stay at or adjacent to the site of administration. For example, by wiping a cotton wipe containing the C3 at 2 mg/mL the off-loaded volume onto the eyelid margin would be about 20 to 40 microliters. This volume would be wicked down the eyelash to the TRPM8 receptors at the transitional epithelium of the eyelids, The blink may further utilize the eyelid “wiper” to distribute the C3 onto the cornea. Overall, the delivery of this small volume is focused only onto the ocular surface.

In some embodiments, the DIPA compound is dissolved in the liquid vehicle at a concentration therein of 0.5 to 5 mg/ml and the liquid vehicle delivers the DIPA compound to the ocular surface.

In some embodiments, the liquid vehicle is an aqueous solution,

In some embodiments, the liquid vehicle is water or isotonic saline.

In some embodiments, the DIPA compound is dissolved in the liquid vehicle at a concentration of 0.5 to 5 mg/ml.

The dosage may range broadly, depending upon the desired effects and the therapeutic indication. The dosage may be a single one or a series of two or more given in the course of one or more days, as is needed by the subject. In some embodiments, the compounds are administered for a period of continuous therapy, for example for a week or more, or for months or years. In some embodiments, a DIPA compound, or a pharmaceutically acceptable salt thereof, can be administered less frequently compared to the frequency of administration of an agent within the standard of care. In some embodiments, a DIPA compound, or a pharmaceutically acceptable salt thereof, can be administered one time per day. In some embodiments, the total time of the treatment regime with a DIPA compound, or a pharmaceutically acceptable salt thereof, can be less compared to the total time of the treatment regime with the standard of care.

As will be understood by those of skill in the art, in certain situations it may be necessary to administer the compounds disclosed herein in amounts that exceed, or even far exceed, the above-stated, preferred dosage range in order to effectively and aggressively treat particularly aggressive diseases or infections.

It should be noted that the attending physician would know how to and when to terminate, interrupt, or adjust administration due to toxicity or organ dysfunctions. Conversely, the attending physician would also know to adjust treatment to higher levels if the clinical response were not adequate (precluding toxicity). The magnitude of an administrated dose in the management of the disorder of interest will vary with the severity of the condition to be treated and to the route of administration. The severity of the condition may, for example, be evaluated, in part, by standard prognostic evaluation methods. Further, the dose and perhaps dose frequency, will also vary according to the age, body weight, and response of the individual patient,

The peripheral sensory nerves of the ocular surface, defined as the epithelia of the cornea, limbus, conjunctiva, and eyelid margins, arise from the ophthalmic division of the trigeminal nerves. The eyelids and cornea have a high density of nerve endings, estimated to be ˜7000 nerve terminals per square millimeter. This density is about 300-600 times that of skin. These nerve endings are initially myelinated but lose myelin as they penetrate the corneal epithelium. The nerve plexus contains ˜80% unmyelinated C fibers and ˜20% myelinated nerve fibers (A-δ fibers). The polymodal nociceptors are 70% C unmyelinated fibers and respond to a large variety of stimuli, including heat, mechanical, endogenous, and exogenous inflammatory stimuli. By contrast, the myelinated A-δ fibers, especially on the eyelid surface at the base of the eyelash hair follicle, code to transmit innocuous cooling.

The complexity and intricacy of the nerve endings in the cornea are illustrated in a recent paper by Schecterson et al. (Molecular Vision 26:576-587, 2020). The nociceptive fibers are associated with the TRP channels called TRPV1 and TRPA1. Also present as separate fiber system is TRPM8. TRPM8 is an integral membrane protein that is a sensor for cooling. Activation of TRPM8 on skin and the aerodigestive tract transduces a site-specific signal of cooling to the brain. The precise physiological role of TRPM8 on the cornea nerve fibers is still unknown.

In a previous study, the inventor of this application has found that the application of a designed, selective TRPM8 agonist called Cryosim-3 (1-diisopropyl-phosphinoylnonane) will relieve eye discomfort in patients with mild to moderate dry eye disease (Yang, J. M.; et al., BMC Ophthalmol 2017, 17, 101.). This is an acute direct antinociceptive action mediated by sensory nerves, much as coolness (e.g., from a cold towel) will reduce discomfort. Now, surprisingly, the inventor of this application has found that Cryosim-3 is also effective for patients with NOP. It should be made clear that a drug's antinociceptive effect does not predict or correlate to an antineuropathic action. For example, opioids (e.g., morphine) and non-steroid anti-inflammatory drugs (NSAIDS, e.g., ibuprofen) are antinociceptive but neither work for neuropathic pain. Neuropathic pain is a chronic condition of 3 months or more. Coolness or cold may aggravate neuropathic pain in conditions such as diabetic ulcers. Therefore, the efficacy of Cryosim-3 in NOP was unusual.

Furthermore, Cryosim-3 treatment of NOP patients appeared to have a disease-modifying effect. The NOP patients were materially improved so that patients had a better quality of life, which persisted even after the termination of Cryosim-3 use. This again was unexpected.

Success in NOP treatment requires that the sensitization process is attenuated; that is, the amplification of a noxious signal is inhibited. Peripheral sensitization and central sensitization can be distinguished by using a local anesthetic eyedrop, e.g., proparacaine hydrochloride solution (Alcaine®). It is known that, although symptoms in some patients could be blocked temporarily (less than 30 min) by alcaine, the NOP was mainly caused by central sensitization. That is, patients with NOP experience persistent ocular surface discomfort and, over time, become preoccupied with the noxious signals and amplify it mentally, especially in the evening hours. This mental preoccupation with the eye discomfort, a sign of central sensitization, exacerbates the NOP.

To treat NOP, the inventor of this application has found that it was important to apply the Cryosim-3 for at least one week on a regular schedule of four times per day (q.i.d.) with an eye wipe. Further improvements were seen when the treatment was extended to one month. This regular application, with patient education on the benefits, was key to achieve clinical improvement.

In summary, a new and effective treatment for NOP has been elucidated based on regular application of Cryosim-3 to the ocular surface for a duration of at least one week. The details of this discovery are in Example 1.

Unless defined otherwise, all technical and scientific terms used herein have the same meaning as is commonly understood by one of ordinary skill in the art. All patents, applications, published applications and other publications referenced herein are incorporated by reference in their entirety unless stated otherwise. In the event that there are a plurality of definitions for a term herein, those in this section prevail unless stated otherwise.

The terminology used herein is for the purpose of describing particular cases only and is not intended to be limiting. As used herein, the singular forms “a,” “an” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in a manner similar to the term “comprising.”

A “subject” refers to an animal that is the object of treatment, observation, or experiment. “Animal” includes cold- and warm-blooded vertebrates and invertebrates such as fish, shellfish, reptiles and, in particular, mammals. “Mammal” includes, without limitation, mice, rats, rabbits, guinea pigs, dogs, cats, sheep, goats, cows, horses, primates, such as monkeys, chimpanzees, and apes, and humans. In some embodiments, the subject is human.

The terms “treating,” “treatment,” “therapeutic,” or “therapy” do not necessarily mean total cure or abolition of the disease or condition. Any alleviation of any undesired signs or symptoms of a disease or condition, to any extent can be considered treatment and/or therapy.

Allodynia: Pain due to a stimulus that does not normally provoke pain.

Analgesia: Absence of pain in response to stimulation which would normally be painful.

Dysesthesia: An unpleasant abnormal sensation, whether spontaneous or evoked.

Hyperalgesia: Increased pain from a stimulus that normally provokes pain.

Neuropathic pain: Pain caused by a lesion or disease of the somatosensory nervous system.

Nociception: The neural process of encoding noxious stimuli-.

Nociceptor: A high-threshold sensory receptor of the peripheral somatosensory nervous system that is capable of transducing and encoding noxious stimuli.

Nociceptive neuron: A central or peripheral neuron of the somatosensory nervous system that is capable of encoding noxious stimuli.

Nociceptive pain: Pain that arises from actual or threatened damage to non-neural tissue and is due to the activation of nociceptors.

Nociceptive stimulus: An actually or potentially tissue-damaging event transduced and encoded by nociceptors.

Nociceptor: A high-threshold sensory receptor of the peripheral somatosensory nervous system that is capable of transducing and encoding noxious stimuli.

Noxious stimulus: A stimulus that is damaging or threatens damage to normal tissues.

Pain threshold: The minimum intensity of a stimulus that is perceived as painful.

Sensitization: Increased responsiveness of nociceptive neurons to their normal input, and/or recruitment of a response to normally subthreshold inputs.

Central sensitization: Increased responsiveness of nociceptive neurons in the central nervous system to their normal or subthreshold afferent input.

Peripheral sensitization: Increased responsiveness and reduced threshold of nociceptive neurons in the periphery to the stimulation of their receptive fields.

### Example 1

This example is a pilot study of topical TRPM8 agonist (cryosim-3) for relieving neuropathic ocular pain in human subjects.

Abstract: Activation of TRPM8, a cold-sensing receptor located on the cornea and eyelid, has the potential to relieve the neuropathic ocular pain (NOP) in dry eye (DE) by inhibiting other aberrant nociceptive inputs. The effect of a topical TRPM8 agonist, cryosim-3 (C3), on relieving DE-associated NOP was investigated. Methods: A prospective pilot study of 15 patients with DE-associated NOP was conducted. These patients applied topical C3 to their eyelid, 4 times/day for 1 month. The patients underwent clinical examinations. They also completed the Ocular Pain Assessment Survey (OPAS), which is a validated questionnaire for NOP, at baseline, 1 week, and 1 month after treatment. Result: At 1 week, the OPAS scores of eye pain intensity, quality of life (driving/watching TV, general activity, sleep, and enjoying life/relations with other people), and associated factors (burning sensation, light sensitivity, and tearing) significantly improved. The total OPAS scores of eye pain intensity, quality of life, and associated factors remained improved at 1 month. The Schirmer test scores also improved at 1 month. Conclusion: TRPM8 agonist (C3) could be a novel agent for treating patients with DE-associated NOP who are unresponsive to conventional treatments.

Dry eye (DE) is a multifactorial disease of the ocular surface characterized by a loss of homeostasis of the tear film and accompanying ocular symptoms [1]. It has a prevalence of 10% to 70% [1]. Some patients with DE experience severe pain that reduces their quality of life (QoL) with minimal ocular signs [1]. Topical agents could be applied as a part of DE treatment to reduce inflammation and tear film osmolality [2]. Generally, if the ocular pain cannot be resolved with topical treatment, other specific causes should be suspected, in particular, neuropathic pain could be the underlying cause [3,4]. In DE, ocular pain disproportionally outweighing the clinical signs is suggestive of underlying neuropathic ocular pain (NOP) nature [4].

Transient receptor potential (TRP) cation channels are associated with the perception of chemical and temperature stimulations [5]. Within the TRP family, TRPM8 is a cold-sensing receptor located on nerve endings of the ophthalmic branch of the trigeminal nerve [6]. Since the activation of TRPM8 can inhibit other aberrant nociceptive inputs, agents for targeting this channel might have the potential to relieve the NOP in DE [7,8]. In particular, TRPM8 is distributed in not only cornea but also eyelid; therefore, it can be activated using topical agents that are applied onto the eyelid without directly instilling eye drops to the cornea [6,9,10]. In our previous study, we revealed the effectiveness of topical cryosim-3 (C3)—a water-soluble and selective TRPM8 agonist—in the treatment of DE by increasing basal tear secretion and alleviating ocular discomfort without any complications [9]. In this pilot study, we aimed to investigate the effect of the topical TRPM8 agonist (C3) on relieving NOP in patients with DE.

### Methods

This prospective non-randomized pilot study was conducted in accordance with the tenets of the Declaration of Helsinki. Ethical approval was obtained from the Chonnam National University Hospital Institutional Review Board (CNUH-2018-274). Informed consent was obtained from all included patients. The sample size was calculated using the G*Power software (version 3.1.9.4; Heinrich-Heine University, Germany) with a level of α=0.05 and a power of 95% to detect a 2-point difference in pain scales. Accordingly, a total sample size of 13 patients was found sufficient.

Patients with DE accompanied by NOP features, who underwent evaluation between January and December in 2018, were enrolled. DE was diagnosed based on OSDI score ≥13 and tear break-up time (TBUT) ≤7 s. The inclusion criteria were as follows: (1) chronic ocular pain which was unresponsive to conventional topical agents (i.e. lubricants, anti-inflammatories, secretagogues, etc.) for >3 months; (2) discordance between the painful DE symptoms and signs accompanying with specific descriptors, including burning or stinging; and (3) a Wong-Baker FACES Pain Rating Scale (WBFPS) score 24. Patients who had a history of ocular diseases other than DE, and those receiving systemic medications that alter the pain and mood statuses were excluded.

The patients were treated with add-on C3 while undergoing conventional topical treatment. C3 samples (2 mg/mL) were diluted in purified water, soaked in gauze, and packaged using automated equipment. The patients applied topical C3 by wiping the gauze on the closed eyelid margin, 4 times/day for 1 month (FIG. 1).

The OSDI questionnaire which ranged from 0 to 100 was used to quantify the vision-related QoL. TBUT (tear breakup time), the time interval between the last complete blink and the first appearance of disruption of the tear film, was measured thrice and the mean value was used for analysis. Corneal staining scores were assessed using the area-density index, by multiplying the area and density score. The Schirmer test score represented the length of wetting, and was measured using a calibrated sterile strip placed at the lateral canthus for 5 min under topical anesthesia (0.5% proparacaine). Only the score of the right eye was assessed.

The WBFPS was chosen to screen the pain severity in the patients with DE. The patients chose the face that best depicted the pain they were experiencing. At baseline, 1 week, and 1 month after treatment, the patients also completed the OPAS which is a validated questionnaire for neuropathic pain as previously described [11]. The questions were divided into sections for analysis: questions 4-9, pertained to eye pain intensity (0 to 60); questions 10-11, pertained to non-eye pain (0 to 20); questions 13-19 (0-10, total score 0 to 60), assessed the QoL (reading and/or computer use, driving and/or watching TV, general activity, mood, sleep, and enjoying life/relations with other people); questions 20-21 (each score 0-1, total score 0-2), assessed aggravating factors (mechanical and chemical stimuli); and questions 22-25 (each score 0-1, total score 0-4), assessed associated factors (redness; burning; sensitivity to light; and tearing). The section on symptomatic relief of the OPAS was excluded, and only questions 4-25 were analyzed. The questions were divided into 5 sections as follows: eye pain intensity, non-eye pain, QoL, aggravating factors, and associated factors.

Statistical analyses were conducted using PASW Statistics for Windows, Version 18.0 (SPSS Inc., Chicago, IL, USA). The normality of distribution was assessed using the Shapiro-Wilk test. The Wilcoxon signed-rank test and repeated-measures analysis of variance with Bonferroni's post-hoc test were used for comparing parameters before and after treatment. A P<0.05 was considered statistically significant.

### Results

This study enrolled 20 patients with DE accompanying NOP features. Five patients (25.0%) discontinued the treatment because of drug ineffectiveness or intolerance. The remaining 15 patients (75.0%) were included in the analysis. Their mean age was 59.5±13.0 years, and nine patients (60.0%) were women. Five patients had a history of intraocular surgery and one patient had a history of ocular trauma,

At 1 week after treatment, eye pain intensity, QoL (driving/watching TV, general activity, sleep, and enjoying life/relations with other people), and associated factors (burning sensation, light sensitivity, and tearing) were improved. The total Ocular Pain Assessment Survey (OPAS) scores of eye pain intensity, QoL (sleep), and associated factors (burning sensation and light sensitivity) remained improved at 1 month. However, the score of non-eye pain and aggravating factors did not change after treatment (Table 1). Among the clinical DE parameters, OSDI and Schirmer test score were improved at 1 month after treatment (Table 2). There were no significant differences in pain scores according to previous medications (Table 3).

TABLE 1
Changes in the Ocular Pain Assessment Survey scores
after the application of cryosim-3 for 1 month

| | | | | P-value* | | |
|---|---|---|---|---|---|---|
| | Baseline^{a} | 1-week^{b} | 1-month^{c} | a vs. b | a vs. c | b vs. c |
| Eye pain | 30.60 ± 12.84 | 26.47 ± 11.45 | 21.53 ± 10.84 | 0.009 | 0.015 | 0.073 |
| intensity | | | | | | |
| (0-60) | | | | | | |
| Non-eye | 7.67 ± 6.22 | 6.73 ± 6.18 | 5.47 ± 5.62 | 0.999 | 0.435 | 0.409 |
| pain | | | | | | |
| (0-20) | | | | | | |
| Quality of | 33.53 ± 14.24 | 27.60 ± 15.49 | 27.17 ± 16.06 | 0.003 | 0.022 | 0.743 |
| life (total | | | | | | |
| 0-60) | | | | | | |
| Reading | 7.79 ± 1.76 | 7.14 ± 2.48 | 6.93 ± 2.59 | 0.120 | 0.054 | 0.272 |
| and/or | | | | | | |
| computer | | | | | | |
| use (0-10) | | | | | | |
| Driving | 6.80 ± 2.31 | 5.27 ± 2.52 | 5.60 ± 2.90 | 0.002 | 0.070 | 0.417 |
| and/or | | | | | | |
| watching | | | | | | |
| TV (0-10) | | | | | | |
| General | 4.00 ± 3.18 | 3.27 ± 2.71 | 3.20 ± 2.86 | 0.016 | 0.138 | 0.843 |
| activity | | | | | | |
| (walking, | | | | | | |
| etc.) (0-10) | | | | | | |
| Mood | 5.40 ± 2.77 | 4.53 ± 2.50 | 4.40 ± 2.47 | 0.121 | 0.177 | 0.769 |
| (0-10) | | | | | | |
| Sleep | 4.27 ± 3.81 | 2.93 ± 3.67 | 2.73 ± 3.81 | 0.027 | 0.049 | 0.486 |
| (0-10) | | | | | | |
| Enjoying | 5.07 ± 2.84 | 4.33 ± 2.97 | 4.27 ± 3.03 | 0.036 | 0.068 | 0.806 |
| life/relations | | | | | | |
| with other | | | | | | |
| people | | | | | | |
| (0-10) | | | | | | |
| Aggravating | 1.11 ± 0.49 | 0.87 ± 0.56 | 0.88 ± 0.57 | 0.113 | 0.132 | 0.077 |
| factors | | | | | | |
| (total 0-2) | | | | | | |
| Mechanical | 0.63 ± 0.29 | 0.47 ± 0.25 | 0.47 ± 0.26 | 0.068 | 0.086 | 0.999 |
| stimuli | | | | | | |
| (0-1) | | | | | | |
| Chemical | 0.47 ± 0.35 | 0.41 ± 0.35 | 0.41 ± 0.32 | 0.363 | 0.432 | 0.872 |
| stimuli | | | | | | |
| (0-1) | | | | | | |
| Associated | 2.09 ± 0.76 | 1.55 ± 0.85 | 1.58 ± 0.93 | 0.006 | 0.046 | 0.835 |
| factors | | | | | | |
| (total 0-4) | | | | | | |
| Redness | 0.41 ± 0.32 | 0.41 ± 0.30 | 0.39 ± 0.30 | 0.094 | 0.104 | 0.080 |
| (0-1) | | | | | | |
| Burning | 0.57 ± 0.37 | 0.40 ± 0.33 | 0.29 ± 0.29 | 0.007 | 0.002 | 0.015 |
| sensation | | | | | | |
| (0-1) | | | | | | |
| Sensitivity | 0.76 ± 0.24 | 0.57 ± 0.26 | 0.59 ± 0.28 | 0.005 | 0.030 | 0.663 |
| to light | | | | | | |
| (0-1) | | | | | | |
| Tearing | 0.36 ± 0.29 | 0.17 ± 0.18 | 0.21 ± 0.27 | 0.013 | 0.197 | 0.578 |
| (0-1) | | | | | | |

All values are presented as mean±SD. *Compared using repeated measures analysis of variance with Bonferroni's post-hoc test.

TABLE 2
Changes in clinical parameters after the
application of cryosim-3 for 1 month

| | | | | P- |
|---|---|---|---|---|
| | Baseline | 1-month | Z | value |
| Ocular surface disease index | 57.5 ± 13.8 | 40.2 ± 12.6 | −3.41 | 0.001 |
| Tear break-up time (s) | 4.13 ± 0.83 | 4.00 ± 0.85 | −0.82 | 0.414 |
| Schirmer test score (mm) | 7.07 ± 2.76 | 8.47 ± 2.80 | −3.02 | 0.003 |
| Corneal staining score (0-9) | 0.60 ± 0.91 | 0.13 ± 0.35 | −1.82 | 0.068 |

All values are presented as mean ± SD. Compared using the Wilcoxon signed rank test.

TABLE 3
Previous medications and Wong-Baker FACES Pain
Rating Scale (WBFPS) score in enrolled patients

| Pa- | | WBFPS score | | |
|---|---|---|---|---|
| tient | | Base- | 1 | 1 |
| No. | Previous medication | line | week | month |
| 1 | HA 0.1%, CsA 0.05%, diquafosol | 9 | 6 | 4 |
| 2 | HA 0.15% | 4 | 1 | 2 |
| 3 | HA 0.15%, 0.1% flumetholone, diquafosol | 7 | 7 | 5 |
| 4 | HA 0.15%, 0.1% flumetholone, diquafosol | 7 | 6 | 6 |
| 5 | HA 0.15%, diquafosol | 6 | 3 | 3 |
| 6 | HA 0.15%, carbomer, 0.1% | 6 | 5 | 5 |
| | flumetholone, diquafosol | | | |
| 7 | HA 0.1%, CsA 0.05%, 0.5% loteprednol, | 6 | 6 | 6 |
| | diquafosol | | | |
| 8 | HA 0.3%, carbomer | 8 | 5 | 4 |
| 9 | HA 0.3%, diquafosol | 6 | 6 | 5 |
| 10 | HA 0.3%, carbomer, diquafosol | 8 | 6 | 4 |
| 11 | HA 0.15%, CsA 0.05%, 0.1% | 4 | 1 | 1 |
| | flumetholone, diquafosol | | | |
| 12 | HA 0.3%, CsA 0.05%, 0.1% | 8 | 7 | 7 |
| | flumetholone | | | |
| 13 | HA 0.15%, 0.1% flumetholone, diquafosol | 7 | 7 | 5 |
| 14 | HA 0.18%, CsA 0.05%, diquafosol | 4 | 1 | 1 |
| 15 | HA 0.3%, carbomer, CsA 0.05%, 0.5% | 4 | 1 | 1 |
| | loteprednol | | | |

| | | 1 | 1 |
|---|---|---|---|
| Mann-Whitney U analysis | Baseline | week | month |
| Previous CsA (yes/no) | 0.607 | 0.456 | 0.456 |
| Previous corticosteroids (yes/no) | 0.694 | 0.281 | 0.121 |
| Previous secretagogues (yes/no) | 0.928 | 0.516 | 0.710 |

HA, hyaluronic acid; CsA, cyclosporin A.

DE is a multifactorial disease of the ocular surface that is accompanied by ocular symptoms [1]. The prevalence of DE has increased considerably worldwide over the last three decades [1]. Some patients with DE experience ocular pain that affects their QoL without any specific abnormal ocular signs [1]. The classification of pain is based on the underlying etiology: (1) nociceptive pain caused by actual or threatened damage to tissues due to the activation of nociceptors, and (2) neuropathic pain caused by a lesion or disease of the somatosensory nervous system [12]. Repeated peripheral nerve injury can lead to peripheral sensitization, and prolonged peripheral ectopic pain initiates central sensitization [4]. Ocular pain symptoms disproportionally outweighing the clinical signs are suggestive of an underlying NOP that might require specific management including systemic treatment [4].

However, chronic NOP associated with DE is a challenging clinical problem that is difficult to treat with conventional medications [4,13]. Conventional topical agents such as cyclosporine A could decrease the release of proinflammatory neuropeptides and cytokines from injured nerves, thereby affecting nociceptive pain and peripheral sensitization [13]. However, these topical treatments appeared to have limitations in produce an improvement in the corneal nerve morphologic status and central sensitization in patients with chronic NOP. Current systemic medication mainly includes oral antidepressants, anticonvulsants, or gabapentinoids; however, these systemic treatments have several limitations, such as delayed onset, variable efficacy, and unacceptable side effects [4,13,14]. In addition, limited data are available to support the use of systemic neuropathic pain medications for NOP associated with DE [14-16]. In this regard, topical agents that are rapid acting, effective, and safe are needed for treating the NOP in DE.

Several members of the TRP super family have emerged as important targets for pain control owing to their critical role in nociception, especially, in chronic states [5]. TRP receptors have been identified in the cornea (TRPV1-4, TRPA1, TRPC4, and TRPM8), conjunctiva (TRPV1, TRPV2, and TRPV4), and eyelid (TRPM8) [6]. In addition, many studies have reported an association between the dysfunction of TRP channels and DE [3,6,17]. TRPM8 is the principal receptor associated with sensing coolness and regulates lacrimal function via response to evaporative cooling and hyperosmolar stimuli [10,18-20]. Several studies have showed that cooling the periocular area with an ice pack or instilling cold artificial tears into the eye could relieve ocular pain after surgery [21,22]. Both TRPM8 agonists and antagonists are considered therapeutic agents for pain control [5-7,23]. TRPM8 antagonists were shown to improve acute and chronic pain such as cold allodynia [23,24]. However, TRPM8 antagonists can reduce basal tear secretion as an undesirable side effect in DE, as shown in the result of experiments using TRPM8 knock-out mice [20]. TRPM8 agonist could present anti-allodynic activity through an excessive activation of TRPM8, leading to its downregulation [25]. It can be seen that these types of animal studies and hypotheses on mechanisms of action, based on TRPM8 agonist or antagonist [23,24] actions at the molecular level, leads to a quagmire of confused thinking. The best answer to treatment of NOP is found on the evidential merit of a clinical trial.

This pilot study showed that the topical application of a TRPM8 agonist (C3) to the eyelid was safe and effective in relieving NOP in patients with DE. We previously showed that the topical application of C3 stimulates basal tear secretion and relieves ocular discomfort in patients with mild DE [9]. The sensory fibers of TRPM8, which innervate the upper eyelid and cornea, are located in the ophthalmic branch of the trigeminal nerve [6]. It was hypothesized in this study that TRPM8 signaling via the eyelid margins may be perceived in the brain as signals from not only the cornea but also the entire ocular surface [9]. Activation of TRPM8 leads to the central synaptic release of glutamate, which then suppresses the injury-activated nociceptive afferent neurotransmission through inhibitory receptors at nerves endings (FIG. 2) [8]. In addition, a hypothesis suggests that these actions attenuate neuropathic sensitization on the dorsal horn [8]. In addition, OSDI and Schirmer test scores improved, but TBUT and corneal staining scores remained unchanged after C3 treatment. TRPM8 agonist is known to increase the basal tear secretion and reduce ocular discomfort via neuronal action, but it does not have direct effect on the tear film [6,9]. These results were consistent with our previous study [9].

Topical delivery of C3 to the eyelid margins could minimize corneal exposure that induces side effects, such as discomfort or paradoxical ocular pain [9]. In addition, the wiping of C3 was more comfortable for patients than conventional instillation of eye drops, and produced painless cooling sensation approximately 40 minutes [9]. The OPAS scores also decreased at 1 week after treatment, indicating that the topical drug produces effect faster than systemic drugs do [14]. Moreover, although the effect was temporary, C3 was particularly effective when the patients experienced severe pain due to DE, such as when driving or sleeping, thereby resulting in an improved QoL.

In addition, patients in this example did not respond to conventional treatment for a long period of time (122.7 days), but they showed an improvement of ocular pain within 1 week after C3 treatment. This improvement suggests a direct effect of C3 treatment rather than a delayed effect of previous conventional treatment. Thus, the TRPM8 agonist (C3) could be a novel agent for treating NOP in patients with DE who are unresponsive to conventional topical treatment.

### REFERENCES

1. Craig, J. P.; Nichols, K. K.; Akpek, E. K.; Caffery, B.; Dua, H. S.; Joo, C.-K.; Liu, Z.; Nelson, J. D.; Nichols, J. J.; Tsubota, K.; et al. TFOS DEWS II Definition and Classification Report. Ocul Surf 2017, 15, 276-283, doi:10.1016/j.jtos.2017.05.008.
2. Jones, L.; Downie, L. E.; Korb, D.; Benitez-Del-Castillo, J. M.; Dana, R.; Deng, S. X.; Dong, P. N.; Geerling, G.; Hida, R. Y.; Liu, Y.; et al. TFOS DEWS II Management and Therapy Report. Ocul Surf 2017, 15, 575-628, doi:10.1016/j.jtos.2017.05.006.
3. Belmonte, C.; Nichols, J. J.; Cox, S. M.; Brock, J. A.; Begley, C. G.; Bereiter, D. A.; Dartt, D. A.; Galor, A.; Hamrah, P.; Ivanusic, J. J.; et al. TFOS DEWS II Pain and Sensation Report. The Ocular Surface 2017, 15, 404-437, doi:10.1016/j.jtos.2017.05.002.
4. Galor, A.; Moein, H.-R.; Lee, C.; Rodriguez, A.; Felix, E. R.; Sarantopoulos, K. D.; Levitt, R. C. Neuropathic Pain and Dry Eye. The Ocular Surface 2018, 16, 31-44, doi:10.1016/j.jtos.2017.10.001.
5. Brederson, J.-D.; Kym, P. R.; Szallasi, A. Targeting TRP Channels for Pain Relief. European Journal of Pharmacology 2013, 716, 61-76, doi:10.1016/j.ejphar.2013.03.003.
6. Yang, J.; Wei, E.; Kim, S.; Yoon, K. TRPM8 Channels and Dry Eye. Pharmaceuticals 2018, 11, 125, doi:10.3390/ph11040125.
7. Fernández-Peña, C.; Viana, F. Targeting TRPM8 for Pain Relief. TOPAINJ 2013, 6, 154-164, doi:10.2174/1876386301306010154.
8. Proudfoot, C. J.; Garry, E. M.; Cottrell, D. F.; Rosie, R.; Anderson, H.; Robertson, D. C.; Fleetwood-Walker, S. M.; Mitchell, R. Analgesia Mediated by the TRPM8 Cold Receptor in Chronic Neuropathic Pain. Current Biology 2006, 16, 1591-1605, doi:10.1016/j.cub.2006.07.061.
9. Yang, J. M.; Li, F.; Liu, Q.; Rüedi, M.; Wei, E. T.; Lentsman, M.; Lee, H. S.; Choi, W.; Kim, S. J.; Yoon, K. C. A Novel TRPM8 Agonist Relieves Dry Eye Discomfort. BMC Ophthalmol 2017, 17, 101, doi:10.1186/s12886-017-0495-2.
10. Wei, E. T. Improving Brain Power by Applying a Cool TRPM8 Receptor Agonist to the Eyelid Margin. Med Hypotheses 2020, 142, 109747, doi:10.1016/j.mehy.2020.109747.
11. Qazi, Y.; Hurwitz, S.; Khan, S.; Jurkunas, U. V.; Dana, R.; Hamrah, P. Validity and Reliability of a Novel Ocular Pain Assessment Survey in Quantification and Monitoring of Corneal and Ocular Surface Pain. Ophthalmology 2016, 123, 1458-1468, doi:10.1016/j.ophtha.2016.03.006.
12. Loeser, J. D.; Treede, R.-D. The Kyoto Protocol of IASP Basic Pain Terminology. Pain 2008, 137, 473-477, doi:10.1016/j.pain.2008.04.025.
13. Dieckmann, G.; Goyal, S.; Hamrah, P. Neuropathic Corneal Pain. Ophthalmology 2017, 124, S34-S47, doi:10.1016/j.ophtha.2017.08.004.
14. Yoon, H.-J.; Kim, J.; Yoon, K. C. Treatment Response to Gabapentin in Neuropathic Ocular Pain Associated with Dry Eye. JCM 2020, 9, 3765, doi:10.3390/jcm9113765.
15. Ongun, N.; Ongun, G. T. Is Gabapentin Effective in Dry Eye Disease and Neuropathic Ocular Pain? Acta Neurol Belg 2019, doi:10.1007/s13760-019-01156-w.
16. Galor, A.; Patel, S.; Small, L. R.; Rodriguez, A.; Venincasa, MJ.; Valido, S. E.; Feuer, W.; Levitt, R. C.; Sarantopoulos, C. D.; Felix, E. R. Pregabalin Failed to Prevent Dry Eye Symptoms after Laser-Assisted in Situ Keratomileusis (LASIK) in a Randomized Pilot Study. J Clin Med 2019, 8, doi:10.3390/jcm8091355.
17. Arcas, J. M.; Gonzdlez, A.; Gers-Barlag, K.; González-González, O.; Bech, F.; Demirkhanyan, L.; Zakharian, E.; Belmonte, C.; Gomis, A.; Viana, F. The Immunosuppressant Macrolide Tacrolimus Activates Cold-Sensing TRPM8 Channels. J Neurosci 2019, 39, 949-969, doi:10.1523/JNEUROSCI.1726-18.2018.
18. Knowlton, W. M.; Palkar, R.; Lippoldt, E. K.; McCoy, D. D.; Baluch, F.; Chen, J.; McKemy, D. D. A Sensory-Labeled Line for Cold: TRPM8-Expressing Sensory Neurons Define the Cellular Basis for Cold, Cold Pain, and Cooling-Mediated Analgesia. J Neurosci 2013, 33, 2837-2848, doi:10.1523/JNEUROSCI.1943-12.2013.
19. Quallo, T.; Vastani, N.; Horridge, E.; Gentry, C.; Parra, A.; Moss, S.; Viana, F.; Belmonte, C.; Andersson, D. A.; Bevan, S. TRPM8 Is a Neuronal Osmosensor That Regulates Eye Blinking in Mice. Nat Commun 2015, 6, 7150, doi:10.1038/ncomms8150.
20. Parra, A.; Madrid, R.; Echevarria, D.; del Olmo, S.; Morenilla-Palao, C.; Acosta, M. C.; Gallar, J.; Dhaka, A.; Viana, F.; Belmonte, C. Ocular Surface Wetness Is Regulated by TRPM8-Dependent Cold Thermoreceptors of the Cornea. Nat Med 2010, 16, 1396-1399, doi:10.1038/nm.2264.
21. Fujishima, H.; Yagi, Y.; Toda, I.; Shimazaki, J.; Tsubota, K. Increased Comfort and Decreased Inflammation of the Eye by Cooling after Cataract Surgery. Am J Ophtholmol 1995, 119, 301-306, doi:10.1016/s0002-9394(14)71171-7.
22. Fujishima, H.; Yagi, Y.; Shimazaki, J.; Tsubota, K. Effects of Artificial Tear Temperature on Corneal Sensation and Subjective Comfort. Cornea 1997, 16, 630-634.
23. De Caro, C.; Russo, R.; Avagliano, C.; Cristiano, C.; Calignano, A.; Aramini, A.; Bianchini, G.; Allegretti, M.; Brandolini, L. Antinociceptive Effect of Two Novel Transient Receptor Potential Melastatin 8 Antagonists in Acute and Chronic Pain Models in Rat. Br J Pharmacol 2018, 175, 1691-1706, doi:10.1111/bph.14177.
24. Fakih, D.; Baudouin, C.; Réaux-Le Goazigo, A.; Mélik Parsadaniantz, S. TRPM8: A Therapeutic Target for Neuroinflammatory Symptoms Induced by Severe Dry Eye Disease. International Journal of Molecular Sciences 2020, 21, 8756, doi:10.3390/ijms21228756.
25. De Caro, C.; Cristiano, C.; Avagliano, C.; Bertamino, A.; Ostacolo, C.; Campiglia, P.; Gomez-Monterrey, I.; La Rana, G.; Gualillo, O.; Calignano, A.; et al. Characterization of New TRPM8 Modulators in Pain Perception. Int J Mol Sci 2019, 20, doi:10.3390/ijms20225544.

1. A method for treating a neuropathic ocular pain disorder in a subject in need thereof, comprising topically applying a therapeutically effective amount of a 1-di-isopropyl-phosphinoyl-alkane (DIPA) compound onto an ocular surface of the subject for at least one week, wherein the DIPA compound is dissolved in a liquid vehicle and the liquid vehicle is adapted for focused delivery of the DIPA compound to the ocular surface.

2. The method of claim 1, wherein the DIPA compound is dissolved in the liquid vehicle at a concentration of 0.5 to 5 mg/ml and the liquid vehicle delivers the DIPA compound to the ocular surface.

3. The method of claim 1, wherein the liquid vehicle is an aqueous solution.

4. The method of claim 1, wherein the liquid vehicle is water or isotonic saline.

5. The method of claim 1, wherein the DIPA compound is dissolved in the liquid vehicle at a concentration of 0.5 to 5 mg/ml.

6. The method of claim 1, wherein the DIPA compound dissolved in the liquid vehicle is delivered to the ocular surface of the subject with a wipe.

7. The method of claim 1, wherein the DIPA dissolved in the liquid vehicle is applied 4 times a day to the ocular surface of the subject.

8. The method of claim 1, wherein the DIPA compound is

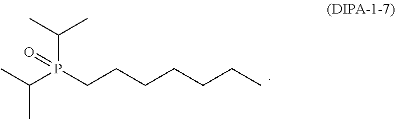

9. The method of claim 1, wherein the DIPA compound is

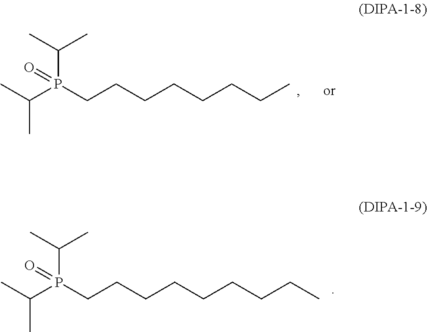

10. The method of claim 1, wherein the DIPA compound is

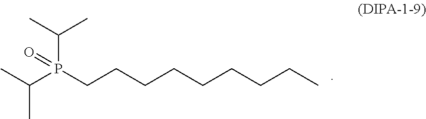 [SMILES: CCCCCCCCCP(=O)(C(C)C)C(C)C]

11. The method of claim 1, wherein the neuropathic ocular pain disorder is caused by dry eye disease or eye surgery.

12. The method of claim 1, wherein the neuropathic ocular pain disorder is caused by trauma to an eye.

13. A topical medication for treating a neuropathic ocular pain disorder in a subject in need, comprising an aqueous solution containing a therapeutically effective amount of a 1-di-isopropyl-phosphinoyl-alkane (DIPA) compound.

14. The topical medication of claim 13, wherein concentration of the DIPA compound in the aqueous solution is 0.5 to 5 mg/mL.

15. The topical medication of claim 13, wherein the DIPA compound is

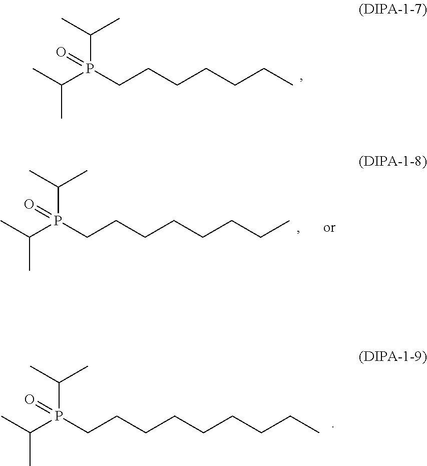

16. The topical medication of claim 13, wherein the neuropathic ocular pain disorder is caused by dry eye disease.

17. The topical medication of claim 13, wherein the neuropathic ocular pain disorder is caused by eye surgery.

18. The topical medication of claim 13, wherein the neuropathic ocular pain disorder is caused by trauma.

19. Use of a 1-di-isopropyl-phosphinoyl-alkane (DIPA) compound for manufacturing a medicament for treating a neuropathic ocular pain disorder in a subject in need thereof, wherein the medicament comprises a therapeutically effective amount of the DIPA compound and a liquid vehicle, wherein the liquid vehicle is adapted for focused delivery of the DIPA compound to an ocular surface of the subject.

20. The use of claim 19, wherein the DIPA compound is [chemical structure]
